SEQUENCE LISTING (26 sequences)

SEQ ID NO: 1 - protein, 5 aa, Artificial sequence
  sequence is synthesized
GGGGS

SEQ ID NO: 2 - protein, 5 aa, Artificial sequence
  sequence is synthesized
QQQQG

SEQ ID NO: 3 - protein, 5 aa, Artificial sequence
  sequence is synthesized
SSSSG

SEQ ID NO: 4 - protein, 17 aa, Artificial sequence
  sequence is synthesized
GSSSSSSSSSSSSSSSG

SEQ ID NO: 5 - protein, 18 aa, Artificial sequence
  sequence is synthesized
GQQQQGQQQQGQQQQGNN

SEQ ID NO: 6 - protein, 7 aa, Artificial sequence
  sequence is synthesized
LSLSPGK

SEQ ID NO: 7 - protein, 8 aa, Artificial sequence
  sequence is synthesized
LSPNRGEC

SEQ ID NO: 8 - protein, 6 aa, Artificial sequence
  sequence is synthesized
LSLSGG

SEQ ID NO: 9 - protein, 7 aa, Artificial sequence
  sequence is synthesized
LSLSPGG

SEQ ID NO: 10 - protein, 15 aa, Artificial sequence
  sequence is synthesized
GGGSGGGGSGGGGSG

SEQ ID NO: 11 - protein, 16 aa, Artificial sequence
  sequence is synthesized
GGGSGGGGSGGGGSGG

SEQ ID NO: 12 - protein, 4 aa, Artificial sequence
  sequence is synthesized
GGGS

SEQ ID NO: 13 - protein, 8 aa, Artificial sequence
  sequence is synthesized
GGGSGGGS

SEQ ID NO: 14 - protein, 12 aa, Artificial sequence
  sequence is synthesized
GGGSGGGSGGGS

SEQ ID NO: 15 - protein, 16 aa, Artificial sequence
  sequence is synthesized
GGGSGGGSGGGSGGGS

SEQ ID NO: 16 - protein, 20 aa, Artificial sequence
  sequence is synthesized
GGGSGGGSGGGSGGGSGGGS

SEQ ID NO: 17 - protein, 10 aa, Artificial sequence
  sequence is synthesized
GGGGSGGGGS

SEQ ID NO: 18 - protein, 15 aa, Artificial sequence
  sequence is synthesized
GGGGSGGGGSGGGGS

SEQ ID NO: 19 - protein, 20 aa, Artificial sequence
  sequence is synthesized
GGGGSGGGGSGGGGSGGGGS

SEQ ID NO: 20 - protein, 25 aa, Artificial sequence
  sequence is synthesized
GGGGSGGGGSGGGGSGGGGSGGGGS

SEQ ID NO: 21 - protein, 137 aa, Mus musculus
MEWSWVMLFLLSGTAGVRSEVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 22 - protein, 128 aa, Mus musculus
MDFQVQIFSFLLISASVILSRGQIVLTQSPAIMSASPGEKVTMTCSASSSIDYIHWYQQKSGTSPKRWIYDTSKLASGVPARFSGSGSGTSYSLTISSMEPEDAATYYCHQRNSYPWTFGGGTRLEIR

SEQ ID NO: 23 - protein, 330 aa, Homo sapiens [sequence omitted]

SEQ ID NO: 24 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 107 aa, Homo sapiens [sequence omitted]

SEQ ID NO: 26 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2012/0282176 A1
(43) Pub. Date: Nov. 8, 2012

(54) Method and Constructs for the pH Dependent Passage of the Blood-brain-barrier

(75) Inventors: Bernd Bohrmann, Riehen (CH); Per-Ola Freskgard, Reinach BL (CH); Adrian Hugenmatter, Zuerich (CH); Erhard Kopetzki, Penzberg (DE); Ekkehard Moessner, Kreuzlingen (CH); Jens Niewoehner, Muenchen (DE); Hadassah Sumum Sade, Penzberg (DE); Pablo Umaña, Wollerau (CH)

(73) Assignee: Roche Glycart AG, Schlieren (CH)

(21) Appl. No.: 13/450,240

(22) Filed: Apr. 18, 2012

(30) Foreign Application Priority Data
Apr. 20, 2011 (EP) EP11163200.6

(51) Int. Cl. A61K 39/395; G01N 33/53; A61K 51/10; A61K 9/127; A61P 25/00; C07K 19/00; C12N 9/96; B82Y 5/00

(52) U.S. Cl. 424/1.49; 424/134.1; 530/387.3; 435/7.21; 435/188; 424/178.1; 530/391.7; 530/391.3; 424/450; 977/773; 977/915

(57) ABSTRACT

Herein is reported a fusion polypeptide comprising i) at least one binding site, e.g. which comprises an antibody heavy chain variable domain and an antibody light chain variable domain, and which binds to an internalizing cell surface receptor, and ii) at least one pharmaceutically active compound, whereby the EC₅₀-value of the binding pair that binds to an internalizing cell surface receptor determined at pH 5.5 is higher than the EC₅₀-value of the same binding pair determined at pH 7.4 and its use for delivering a pharmaceutically active compound across the blood-brain-barrier.

### RELATED APPLICATIONS

This application claims the benefit of European Patent Application No. 11163200.6 filed on Apr. 20, 2011, which is incorporated by reference herein in its entirety.

### BACKGROUND

Herein is reported a fusion polypeptide comprising at least one binding site and at least one pharmaceutically active compound, whereby the EC₅₀-value of the binding site that binds to an internalizing cell surface receptor determined at pH 5.5 is higher than the EC₅₀-value of the same binding site determined at pH 7.4, and its use for delivering a pharmaceutically active compound across the blood-brain-barrier.

### BACKGROUND OF THE INVENTION

Endothelial or epithelial cell layers interconnected by tight junctions represent a major hurdle for the diffusion of large, polar molecules, especially proteins, into the tissues behind these barriers. While small molecules can be transported across these barriers by specialized channel proteins, the transport mechanisms for proteins are still incompletely understood, but the physiologically most important mechanism is thought to be receptor-mediated transcytosis (RMT).

During RMT a protein ligand binds to a receptor expressed on the luminal side of the barrier cells, which is then internalized by endocytosis. Sorting of endosomal content is achieved in specialized vesicular compartments and depends on signals encoded by the receptor sequence, which mediate trafficking of the receptor into recycling, degradation, or transcytosis pathways. One of the best known examples of RMT is the transport of IgG across intestinal epithelial cells by the neonatal Fc receptor in rodents.

Also for the blood-brain-barrier (BBB), which consists of tightly sealed brain endothelial cells surrounded by pericytes and astrocytes, several RMT pathways have been described, especially the receptors for transferrin, insulin, or low-density lipoprotein. The ligands of these receptors have been shown to harbor properties facilitating transcytosis, one of these properties being pH-dependent binding to their receptors. Insulin, for example, is released from its receptor upon acidification of the endosomal content after internalization.

Researchers have attempted to exploit transcytosis of receptors for the delivery of therapeutic molecules over the blood-brain-barrier by coupling therapeutics to antibodies against these receptors. However, none of these antibodies has been used in a marketed drug yet, probably because of their insufficient transport potential. In fact, unequivocal demonstration of functional therapeutic protein transcytosis over the BBB, shown independently in more than one pharmacodynamic model, is still missing.

FcRn-mediated transcytosis of immunoglobulin G in human renal proximal tubular epithelial cells is reported by Kobayashi, et al. (Kobayashi, N., et al., Am. J. Physiol. Renal. Physiol. 282 (2002) F358-F365). Weksler, B. B., et al. report in FASEB J. 19 (2005) 1872-1874 blood-brain barrier-specific properties of a human adult brain endothelial cell line.

In U.S. Pat. No. 6,030,613 the receptor specific transepithelial transport of immunogens is reported. In WO 02/060919 are reported molecules with extended half-lives, compositions and uses thereof. A process for the preparation of transferrin receptor specific antibody-neuropharmaceutical or diagnostic agent conjugates is reported in WO 93/010819. In WO 2008/119096 transcytotic immunoglobulin are reported. A human blood brain barrier model is reported in WO 2006/056879.

In EP 1 975 178 is reported a transcytotic modular antibody. Blood-brain barrier targeting antibodies are reported in US 2008/019984. Friden, P. M., et al., report the characterization, receptor mapping and blood-brain barrier transcytosis of antibodies to the human transferrin receptor (J. Pharmacol. Exp. Therap. 278 (1996) 1491-1498). Pardridge et al. (Pharm. Res. 12 (1995) 807-816) report that human insulin receptor monoclonal antibody undergoes high affinity binding to human brain capillaries in vitro and rapid transcytosis through the blood-brain barrier in vivo in the primate.

### SUMMARY OF THE INVENTION

Herein is reported that a pH-dependent binding mode enables antibodies directed against internalizing cell surface receptors, especially transcytosis receptors, to efficiently cross a tight layer of barrier cells, especially the blood-brain-barrier. It is shown that an antibody, e.g. binding to the human transferrin receptor as an example of an internalizing cell surface receptor, which has a low binding affinity at pH 5.5 (higher EC₅₀ value) as compared to its affinity at pH 7.4 (lower EC₅₀ value), is transcytosed through blood-brain-barrier endothelial cells, whereas a different antibody showing about equal affinity (binding efficiency, and, thus, EC₅₀ values) at both pH values, is degraded inside the cells. Thus, the EC₅₀-value of the binding site that binds to an internalizing cell surface receptor determined at pH 5.5 is higher (bigger) than the EC₅₀-value of the same binding site determined at pH 7.4. This allows the generation and selection of antibodies against transcytosis receptors that are not intracellularly degraded in endothelial or epithelial barrier cells due to a modified sorting behavior caused by pH-dependent, reversible binding to those receptors.

Herein is reported as one aspect a fusion polypeptide comprising
at least one binding site, which binds to an internalizing cell surface receptor, and
at least one effector moiety,
whereby the EC₅₀-value of the binding site that binds to an internalizing cell surface receptor determined at pH 5.5 is higher than the EC₅₀-value of the same binding site determined at pH 7.4.

In one embodiment the fusion polypeptide is characterized in that the ratio of i) the EC₅₀-value of the binding site that binds to an internalizing cell surface receptor determined at pH 5.5 and ii) the EC₅₀-value of the same binding site to the same receptor determined at pH 7.4 is at least 5. In one embodiment the ratio is 10 or more. In one embodiment the ratio is 15 or more. In one embodiment the ratio is about 15.

In one embodiment the EC₅₀-value of the binding site that binds to an internalizing cell surface receptor determined at pH 5.5 is at least 5-times the EC₅₀-value of the same binding site to the same receptor determined at pH 7.4. In one embodiment the EC₅₀-value determined at pH 5.5 is at least 10-times the EC₅₀-value determined at pH 7.4. In one embodiment the EC₅₀-value determined at pH 5.5 is about 15-times the EC₅₀-value determined at pH 7.4.

In one embodiment the effector moiety is a label, or a cytotoxin, or an enzyme, or a growth factor, or a transcription factor, or a drug, or a radionuclide, or a ligand, or an antibody, or antibody fragment, or a liposome, or a nanoparticle, or a viral particle, or a cytokine

In one embodiment the effector moiety is a pharmaceutically active compound. In one embodiment the pharmaceutically active compound is an anti-Abeta antibody, or an anti-tau antibody, an anti-alpha synuclein antibody, or an active fragment thereof.

In one embodiment the effector moiety is a pharmaceutically active compound that is attached to the fusion polypeptide by a linker. In another embodiment the effector moiety is a pharmaceutically active compound that is directly fused to the fusion polypeptide.

In one embodiment the binding site that binds to an internalizing cell surface receptor has an EC₅₀-value determined at pH 5.5 of 100 ng/ml or more, or of 500 ng/ml or more, or of 1000 ng/ml or more.

In one embodiment the binding site that binds to an internalizing cell surface receptor has an EC₅₀-value determined at pH 7.4 of 100 ng/ml or less, or of 85 ng/ml or less, or of 70 ng/ml or less.

In one embodiment the binding site is a binding pair, which comprises an antibody heavy chain variable domain and an antibody light chain variable domain. In one embodiment the binding pair is selected from an Fv, a Fab, a Fab′, a Fab′-SH, a F(ab′)₂, a diabody, a linear antibody, a single-chain antibody molecule, and a multispecific antibody formed from antibody fragments, a full length heavy chain, a full length light chain, a complete antibody, a bispecific antibody, a trispecific antibody, a tetraspecific antibody, or a hexaspecific antibody. In one embodiment the binding pair is a complete monoclonal antibody. In one embodiment binding pair is at least a fragment of a complete antibody, a member of the immunoglobulin superfamily, or a polypeptide with immunoglobulin-like structure, that retains the binding specificity for its antigen.

In one embodiment the binding site is selected from fibronectin, TCR, CTLA-4, single-chain antigen receptor, e.g. related to T-cell receptor, antibody mimetic, transferrin, apolipoprotein, adnectin, molecules based on anticalin, phylomer, avimer, affibody, ankyrin repeat, Kunitz domain, PDZ-domain, scorpio toxin immunity protein, Knottin, Versabody, Green Fluorescent Protein, and other non-antibody protein scaffolds with binding properties.

In one embodiment the internalizing cell surface receptor is selected from a sialoglycoprotein receptor, an alpha(2,3)sialoglycoprotein receptor, a diphtheria toxin receptor a heparin-binding epidermal growth factor-like growth factor, a folate receptor, a glutamate receptor, a glutathione receptor, an insulin receptor, an insulin-like growth factor receptor, a leptin receptor, a low-density lipoprotein receptor, an LDL-related protein 1 receptor, an LRP2 receptor, an LRP4 receptor, an LRP5 receptor, an LRP6 receptor, an LRP8 receptor, a mannose 6-phosphate receptor, a scavenger receptor (class A or B, types I, II or III, or CD36 or CD163), a substance P receptor, a thiamine transporter, a transferrin-1 receptor, a transferrin-2 receptor, and a vitamin B12 receptor. In one embodiment the internalizing cell surface receptor is a transferrin receptor.

Herein is reported as one aspect a nucleic acid encoding the fusion polypeptide as reported herein.

Herein is reported as one aspect a host cell comprising the nucleic acid as reported herein.

Herein is reported as one aspect a method of producing a fusion polypeptide comprising culturing the host cell as reported herein so that the fusion polypeptide is produced.

Herein is reported as one aspect a pharmaceutical formulation comprising the fusion polypeptide as reported herein and optionally a pharmaceutically acceptable carrier.

Herein is reported as one aspect the fusion polypeptide as reported herein for use as a medicament.

Herein is reported as one aspect the fusion polypeptide as reported herein for use in treating a CNS-related disease.

Herein is reported as one aspect the fusion polypeptide as reported herein for use in delivering a pharmaceutically active compound across the blood-brain-barrier.

Herein is reported as one aspect the use of the fusion polypeptide as reported herein in the manufacture of a medicament.

In one embodiment the medicament is for treatment of a CNS-related disease.

Herein is reported as one aspect a method of treating an individual having a CNS-related disease comprising administering to the individual an effective amount of the fusion polypeptide as reported herein.

Herein is reported as one aspect a method of delivering a pharmaceutically active compound across the blood-brain-barrier in an individual comprising administering to the individual an effective amount of the fusion polypeptide as reported herein to deliver a pharmaceutically active compound across the blood-brain-barrier.

Herein is reported as one aspect a method of delivering a pharmaceutically active compound across the blood-brain-barrier to a subject's brain, comprising administering a pharmaceutically active compound fused to a binding pair, which comprises an antibody heavy chain variable domain and an antibody light chain variable domain, and which binds to an internalizing cell surface receptor, whereby the EC₅₀-value of the binding pair that binds to an internalizing cell surface receptor determined at pH 5.5 is higher than the EC₅₀-value of the same binding pair determined at pH 7.4. In one embodiment the fusion polypeptide is characterized in that the ratio of the EC₅₀-value of the binding site that binds to an internalizing cell surface receptor determined at pH 5.5 and the EC₅₀-value of the same binding site to the same receptor determined at pH 7.4 is at least 5. In one embodiment the ratio is 10 or more. In one the ratio is 15 or more. In also an embodiment the ratio is about 15.

In one embodiment the EC₅₀-value of the binding pair that binds to an internalizing cell surface receptor determined at pH 5.5 is at least 5-times the EC₅₀-value of the same binding pair to the same receptor determined at pH 7.4. In one embodiment the EC₅₀-value determined at pH 5.5 is at least 10-times the EC₅₀-value determined at pH 7.4. In one embodiment the EC₅₀-value determined at pH 5.5 is about 15-times the EC₅₀-value determined at pH 7.4.

Herein is reported as one aspect the use of a fusion polypeptide as reported herein for the delivery of a pharmaceutically active compound across the blood-brain-barrier.

Herein is reported as one aspect a method of transcytosing epithelial cells of a subject comprising administering to the subject a fusion polypeptide as reported herein.

Herein is reported as one aspect a method of increasing transport of at least one pharmaceutically active compound across the blood-brain-barrier in an individual relative to the transport across the blood-brain-barrier of an unconjugated form of the one or more pharmaceutically active compound, comprising administering to the individual an effective amount of a fusion polypeptide as reported herein such that the fusion polypeptide transports the pharmaceutically active compound across the blood-brain-barrier.

Herein is reported as one aspect a method for selecting an antibody or a fusion polypeptide comprising at least one binding site wherein the EC₅₀-value of the antibody or fusion polyp eptide for binding to an internalizing cell surface receptor determined at pH 5.5 is higher than the EC₅₀-value of the same antibody or the same fusion polypeptide to the same receptor determined at pH 7.4.

In one embodiment the antibody or the fusion polypeptide is characterized in that the ratio of the EC₅₀-value of the binding site that binds to an internalizing cell surface receptor determined at pH 5.5 and the EC₅₀-value of the same binding site to the same receptor determined at pH 7.4 is at least 5. In one embodiment the ratio is 10 or more. In one embodiment the ratio is 15 or more. In one embodiment the ratio is about 15.

In one embodiment the EC₅₀-value of the binding site that binds to an internalizing cell surface receptor determined at pH 5.5 is at least 5-times the EC₅₀-value of the same binding site to the same receptor determined at pH 7.4. In one embodiment the EC₅₀-value determined at pH 5.5 is at least 10-times the EC₅₀-value determined at pH 7.4. In one embodiment the EC₅₀-value determined at pH 5.5 is about 15-times the EC₅₀-value determined at pH 7.4.

Herein is reported as one aspect a method for selecting a binding pair for use in efficient blood-brain-barrier transport of one or more pharmaceutically active compounds comprising measuring a ratio of the EC₅₀-values for binding of one or more binding pairs to an internalizing cell surface receptor at pH 5.5 and pH 7.4, and selecting one or more binding pairs wherein the ratio is 10 or more.

In one embodiment of all the aspects as reported herein the CNS-related disease is selected from (i) neurodegenerative diseases or disorders such as Parkinson's disease, Alzheimer's disease, or Huntington's disease, or (ii) psychiatric diseases such as depression, anxiety disorders, schizophrenia, or (iii) neuroinflammatory and other neurological disorders such as multiple sclerosis, Amyotrophic Lateral Sclerosis, autism, or pain, or (iv) tumors of the CNS, or (v) viral and bacterial infections of the CNS.

### DETAILED DESCRIPTION OF THE INVENTION

The present invention demonstrates that a pH-dependent binding mode enables fusion polypeptides comprising at least one binding site and antibodies directed against transcytosis receptors to efficiently cross a tight layer of barrier cells. It is shown for example that an antibody against the human transferrin receptor, which has a low binding affinity at pH 5.5 as compared to its affinity at pH 7.4, is transcytosed through blood-brain barrier endothelial cells, whereas another antibody showing equally efficient binding at both pH values to the transferrin receptor, is degraded inside the cell. The invention allows the selection and generation of antibodies against transcytosis receptors that avoid intracellular degradation in endothelial or epithelial barrier cells by a modified sorting behavior caused by pH-dependent, reversible binding to those receptors.

### I. Definitions

The term “affinity” denotes the strength of the sum total of non-covalent interactions between a single binding site of a molecule (e.g. a polypeptide or an antibody) and its binding partner (e.g. a target or an antigen). Unless indicated otherwise, as used herein, “binding affinity” refers to intrinsic binding affinity which reflects a 1:1 interaction between members of a binding pair (e.g. in a polypeptide-polynucleotide-complex, or between a polypeptide and its target, or between an antibody and its antigen). The affinity of a molecule X for its partner Y can generally be represented by the dissociation constant (Kd). Affinity can be measured by common methods known in the art, such as surface plasmon resonance and also including those reported herein. A higher affinity of a molecule X for its binding partner Y can be seen by a lower Kd and/or EC₅₀ value.

The term “antibody” encompasses the various forms of antibody structures including whole antibodies and antibody fragments. The antibody as reported and used herein can be a human antibody, a humanized antibody, a chimeric antibody, or a T cell antigen depleted antibody. The term “antibody” refers to a protein consisting of one or more polypeptide(s) substantially encoded by immunoglobulin genes. The recognized immunoglobulin genes include the different constant region genes as well as the myriad immunoglobulin variable region genes. Immunoglobulins may exist in a variety of formats, including, for example, Fv, Fab, and F(ab)₂ as well as single chains (scFv) or diabodies. A full length antibody in general comprises two so called light chain polypeptides (light chain) and two so called heavy chain polypeptides (heavy chain). Each of the heavy and light chain polypeptides contains a variable domain (variable region) (generally the amino terminal portion of the polypeptide chain) comprising binding regions that are able to interact with an antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (Clq).

The variable domain of an immunoglobulin's light or heavy chain in turn comprises different segments, i.e. four framework regions (FR) and three hypervariable regions (CDR).

The term “binding pair” denotes a polypeptide, which comprises an antibody heavy chain variable domain and an antibody light chain variable domain. The variable domains can be connected to each other by any suitable means such as a peptide bond, a linker, or a linking non-peptidic component. In one embodiment the binding pair is selected from Fv, Fab, Fab′, Fab′-SH, F(ab′)₂, diabody, linear antibodies, single-chain antibody molecules, and multispecific antibodies formed from antibody fragments, full length heavy chain, full length light chain, complete antibody, bispecific antibody, trispecific antibody, tetraspecific antibody, or hexaspecific antibody. In one embodiment the binding pair is a monoclonal antibody. In one embodiment binding pair is at least a fragment of a complete antibody, a member of the immunoglobulin superfamily, or a polypeptide with immunoglobulin-like structure, that retains the binding specificity for its antigen.

The term “binding site” denotes a polypeptide that can specifically bind to another polypeptide. In one embodiment the binding site is a binding pair. In one embodiment the binding site is a polypeptide with immunoglobulin-like modular structure, which can be selected from the group consisting of fibronectin, TCR, CTLA-4, single-chain antigen receptors, e.g. those related to T-cell receptors and antibodies, antibody mimetics, transferrin, apolipoprotein, adnectins, molecules based on anticalins, phylomers, avimers, affibodies, ankyrin repeats, Kunitz domains, PDZ-domains, scorpio toxins immunity proteins, Knottins, Versabodies, Green Fluorescent Protein and other non-antibody protein scaffolds with antigen binding properties.

The term “CNS-related disease” denotes a disease or disorder of the central nervous system (CNS). CNS-related diseases are, without being limited to, particularly (i) neurodegenerative diseases or disorders such as Parkinson's disease, Alzheimer's disease, or Huntington's disease, (ii) psychiatric diseases such as depression, anxiety disorders, schizophrenia, (iii) neuroinflammatory and other neurological disorders such as multiple sclerosis, Amyotrophic Lateral Sclerosis, autism, or pain; (iv) tumors of the CNS, or (v) viral and bacterial infections of the CNS.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamylamines including altretamine, triethylenemelamine, trietylenephosphoramide, triethylenethiophosphoramide and trimethylomelamine; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitroureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rh6ne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-II; 35 topoisomerase inhibitor RFS 2000; difluoromethylornithine (DMFO); retinoic acid; esperamicins; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

An “anti-angiogenic agent” refers to a compound which blocks, or interferes with to some degree, the development of blood vessels. The anti-angiogenic agent may, for instance, be a small molecule or an antibody that binds to a growth factor or growth factor receptor involved in promoting angiogenesis. The anti-angiogenic factor is in one embodiment an antibody that binds to Vascular Endothelial Growth Factor (VEGF).

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-a and -P; mullerian-inhibiting substance; mouse gonadotropin-associated peptide; inhibin; activin; vascular endothelial growth factor; integrin; thrombopoietin (TPO); nerve growth factors such as NGF-p; platelet growth factor; transforming growth factors (TGFs) such as TGF-a and TGF-p; insulin-like growth factor-I and -II; erythropoietin (EPO); osteoinductive factors; interferons such as interferon-a, -P, and -y; colony stimulating factors (CSFs) such as macrophage-CSF (M-CSF); granulocyte-macrophage-CSF (GM-CSF); and granulocyte-CSF (GCSF); interleukins (ILs) such as IL-I, IL-1a, IL-2, IL-3, IL-4, IL-5, IL-6, IL-7, IL-8, IL-9, IL-IO, IL-II, IL-12; a tumor necrosis factor such as TNF-α or TNF-P; and other polypeptide factors including LIF and kit ligand (KL). As used herein, the term cytokine includes proteins from natural sources or from recombinant cell culture and biologically active equivalents of the native sequence cytokines

The term “fMLP” denotes the tripeptide consisting of N-formylmethionine, leucine and phenylalanine In one embodiment the effector moiety is fMLP or a derivative thereof.

The term “fusion polypeptide” denotes a polypeptide that comprises or is consisting of at least two discrete peptides or polypeptides that are not found together in this way in a polypeptide in nature, i.e. these portions are not occurring in the same polypeptide or in the same order in nature. The portions of the fusion polypeptide are linked by a peptide bond.

The term “peptidic linker” denotes linkers of natural and/or synthetic origin comprising amino acid residues connected to each other via peptide bonds. They consist of a linear amino acid chain wherein the 20 naturally occurring amino acids are the monomeric building blocks. The chain has a length of from 1 to 50 amino acid residues, in one embodiment between 3 and 28 amino acid residues, in a further embodiment between 4 and 20 amino acid residues. The linker may contain repetitive amino acid sequences or sequences of naturally occurring polypeptides. The linker has the function to ensure that the two components connected through the linker can fold correctly and be presented properly due to steric and rotational freedom. In one embodiment the linker is a “synthetic peptidic linker” that is designated to be rich in glycine, glutamine, and/or serine residues. These residues are arranged e.g. in small repetitive units of up to five amino acids, such as (G)GGGS, (Q)QQQG, or (S)SSSG (SEQ ID NO: 1, 2, and 3). This small repetitive unit may be repeated for two to five times to form a multimeric unit. Other synthetic peptidic linkers are composed of a single amino acid, that is repeated between 10 to 20 times, such as e.g. serine in the linker GSSSSSSSSSSSSSSSG (SEQ ID NO: 4). In one embodiment the linker is selected from [GQ₄]₃GNN (SEQ ID NO: 5), LSLSPGK (SEQ ID NO: 6), LSPNRGEC (SEQ ID NO: 7), LSLSGG (SEQ ID NO: 8), LSLSPGG (SEQ ID NO: 9), G₃[SG₄]₂SG (SEQ ID NO: 10), or G₃[SG₄]₂SG₂ (SEQ ID NO: 11).

The term “prodrug” refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella, et al., “Prodrugs: A Chemical Approach to Targeted Drug Delivery,” Directed Drug Delivery, Borchardt et al., (ed.), pp. 247-267, Humana Press (1985). The prodrugs that can be used as effector moiety include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described herein.

The term “cytotoxic moiety” refers to a substance that inhibits or prevents a cellular function and/or causes cell death or destruction. Cytotoxic agents include, but are not limited to radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed herein.

An “effective amount” of an agent, e.g., a pharmaceutical formulation, refers to an amount effective, at dosages and for periods of time necessary, to achieve the desired therapeutic or prophylactic result.

The term “EC₅₀-value” denotes the half maximal effective concentration of a polypeptide, e.g. an antibody, that induces a response of 50% between the baseline value and the maximum value in a determination system, e.g. an ELISA. This is a measure of a therapeutic drug's potency. Thus, the EC₅₀-value is the concentration that is calculated based on experimental data corresponding to the concentration of a drug substance resulting in 50% effect. Decreasing EC₅₀-values denotes a higher affinity and potency of the drug.

A “human antibody” is an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g. CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “immunoconjugate” is an antibody or antibody fragment conjugated to one or more non antibody derived molecules, including but not limited to a member of a binding pair, a nucleic acid, or an effector moiety.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “internalizing cell surface receptor” denotes a group of cell surface receptors comprising at least the following members: asialoglycoprotein receptors, alpha(2,3)sialoglycoprotein receptor, diphtheria toxin receptor (DTR, which is the membrane-bound precursor of heparin-binding epidermal growth factor-like growth factor (HB-EGF)), folate receptor, glutamate receptors, glutathione receptor, insulin receptor, insulin-like growth factors (IGF) receptors, leptin receptors, low-density lipoprotein (LDL) receptor, LDL-related protein 1 receptor (LRP1, type B), LRP2 receptor (also known as megalin or glycoprotein 330), LRP4 receptor, LRP5 receptor, LRP6 receptor, LRP8 receptor, mannose 6-phosphate receptor, scavenger receptors (class A or B, types I, II or III, or CD36 or CD163), substance P receptor, thiamine transporter, transferrin-1 and -2 receptors, and vitamin B12 receptors.

The term “monoclonal antibody” refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies or monoclonal antibody fragments to be used in the fusion polypeptide as reported herein may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “transcellular transport” denotes a multistep process in which a molecule, especially a macromolecule or biopolymer such as an antibody, is transported across the cytosol of a cell. In the first step of a transcellular transport material/molecules of the extracellular space or cell surface-associated or -bound molecules are enclosed in a vesicle. This step is called endocytosis. The vesicle diffuses across the cytosol of the cell. Thereafter the endocytotic step is reversed, i.e. the vesicle is fused with the cell membrane and the interior of the vesicle is released to the extracellular space. Transcellular transport takes place for example in epithelial cells, cells of the blood-brain-barrier, neurons, or intestinal cells.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, fusion polypeptides as reported herein are used to delay development of a disease or to slow the progression of a disease.

The term “variable region” or “variable domain” refers to the domain of an antibody heavy or light chain that is involved in binding the antibody to its antigen. The variable domains of the heavy chain and light chain (VH and VL, respectively) of a native antibody generally have similar structures, with each domain comprising four conserved framework regions (FRs) and three hypervariable regions (HVRs) (see, e.g., Kindt, T. J., et al., Kuby Immunology, 6^{th} ed., W.H. Freeman and Co., N.Y. (2007), page 91). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (see, e.g., Portolano, S., et al., J. Immunol. 150 (1993) 880-887, Clackson, T., et al., Nature 352 (1991) 624-628).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The articles “a” and “an” are used herein to refer to one or to more than one (i.e., to at least one) of the grammatical object of the article. By way of example, “an antibody” means one antibody or more than one antibody.

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

The term “specifically binding” denotes that the binding site or polypeptide or antibody or antibody fragments binds to its target with an dissociation constant (Kd) of 10⁻⁵ M or less, in one embodiment of from 10⁻⁷ M to 10⁻¹³ M, in a further embodiment of from 10⁻⁷ M to 10⁻⁹ M. The term is further used to indicate that the polypeptide does not bind to other biomolecules present, i.e. it binds to other biomolecules with a dissociation constant (Kd) of 10⁻⁴ M or more, in one embodiment of from 10⁻⁴M to 1 M.

The term “pharmaceutically active compound” denotes any molecule or combination of molecules whose activity it is desired to be delivered to a place of action. Pharmaceutically active compounds include, but are not limited to drugs (e.g. polypeptides, antibodies), labels, cytotoxins (e.g. Pseudomonas exotoxin, ricin, abrin, Diphtheria toxin, and the like), enzymes, growth factors, transcription factors, radionuclides, ligands, liposomes, nanoparticles, viral particles, cytokines, and the like.

### II. Compositions and Methods

Herein is reported a fusion polypeptide with which it is possible to transport therapeutics (biologically active compounds) such as polypeptides, antibodies, or toxins, across a cell membrane, especially the blood-brain-barrier. The fusion polypeptide as reported herein employs therefore a general transport mechanism, i.e. receptor-mediated endocytosis and transcytosis using an internalizing cell surface receptor.

Herein is reported as one embodiment a fusion polypeptide comprising
at least one binding site, which binds to an internalizing cell surface receptor, and
at least one pharmaceutically active compound,
whereby the ratio of the EC₅₀-value of the binding site that binds to an internalizing cell surface receptor determined at pH 5.5 and the EC₅₀-value of the same binding site to the same receptor determined at pH 7.4 is 10 or more.

In one embodiment the ratio is 15 or more.

In one embodiment the ratio is 100 or less.

In one embodiment the ratio is 10 to 100.

In one embodiment the binding site has an EC₅₀-value determined at pH 5.5 of 700 ng/ml or more. In one embodiment the binding site has an EC₅₀-value determined at pH 5.5 of 850 ng/ml or more. In one embodiment the binding site has an EC₅₀-value determined at pH 5.5 of 1000 ng/ml or more.

In one embodiment the binding site is a binding pair, which comprises an antibody heavy chain variable domain and an antibody light chain variable domain. In one embodiment the binding pair is selected from Fv, Fab, Fab′, Fab′-SH, F(ab′)₂, diabody, linear antibodies, single-chain antibody molecules, and multispecific antibodies formed from antibody fragments, full length heavy chain, full length light chain, complete antibody, bispecific antibody, trispecific antibody, tetraspecific antibody, or hexaspecific antibody. In one embodiment the binding pair is a monoclonal antibody. In one embodiment the binding pair is at least a fragment of a complete antibody, a member of the immunoglobulin superfamily, or a polypeptide with immunoglobulin-like structure, that retains the binding specificity for its antigen.

In one embodiment the binding site is selected from fibronectin, TCR, CTLA-4, single-chain antigen receptors, e.g. those related to T-cell receptors and antibodies, antibody mimetics, transferrin, apolipoprotein, adnectins, molecules based on anticalins, phylomers, avimers, affibodies, ankyrin repeats, Kunitz domains, PDZ-domains, scorpio toxins immunity proteins, Knottins, Versabodies, Green Fluorescent Protein and other non-antibody protein scaffolds with binding properties.

In one embodiment the binding site is a full length antibody or antibody fragment that specifically binds to the transferrin receptor.

Without being bound by theory FIG. 1 shows a schematic drawing of the pH-dependent transcytosis mechanism. Iron-loaded holo-transferrin (middle panel) is endocytosed with the transferrin receptor from the apical membrane of the brain endothelial cells. Upon endosomal acidification, iron is released from the holo-transferrin, initiated by a conformational change in transferrin-binding domain of the receptor. Apo-transferrin remains bound to the receptor. After passing through the sorting endosome, the receptor is either recycled to the apical membrane or transcytosed to the basolateral membrane. After vesicle-membrane fusion, apo-transferrin, which has no affinity for the receptor at pH 7.4, dissociates from the receptor and leaves the cell. In contrast (left panel), the transferrin-receptor antibody mAb 128.1, which binds the receptor with high affinity at pH 7.4 as well as at pH 5.5, i.e. has an EC₅₀ value ratio of less than 5 (1.3), forms a tight complex with the receptor that is also stable at the pH value in the endosome. The presence of the pH-stable complex prevents recycling and transcytosis, but rather induces re-routing of the receptor into CD63-positive late endosomes. Anti-transferrin-receptor antibodies with a pH-dependent binding profile (exemplified by antibody MEM-189, which shows reduced receptor binding at reduced (acid) pH values as at endosomal pH; right panel, EC₅₀ value ratio of more than 10 (15.6)) can undergo transcytosis and recycling, without being bound by theory probably by reversible, low affinity interaction with the transferrin-receptor in the endosomal compartments.

In FIG. 3 the validation of the transcytosis assay as used herein by transcytosis of ¹²⁵I-transferrin through hCMEC/D3 brain endothelial cells is shown. hCMEC/D3 cells on collagen-coated filter inserts were loaded with ¹²⁵I-labeled transferrin for one hour. Afterwards the inserts were washed and transferred to a new plate at 37° C. (FIG. 3A) or 4° C. (FIG. 3B). At the indicated time points, radioactivity in the cell lysates (black squares), the apical (grey columns) and basolateral (white columns) medium compartments was measured by gamma counting (CPM) after TCA precipitation. The sum of the radioactivity values for each time point is shown in white triangles. While at 37° C., transferrin is leaving the cell layer at equal amounts into apical or basolateral medium compartments (A), it stays inside the cells at 4° C. (B), demonstrating that the transport process is energy-dependent.

In FIG. 4 it is shown that mAb 128.1 against the human transferrin receptor does not leave hCMEC/D3 cells. ¹²⁵I-labeled mAb 128.1 was allowed to be taken up by hCMEC/D3 cells and radioactivity was determined in cellular, apical and basolateral medium compartments as described above (FIG. 3). No intact antibody leaves the cells into the apical or basolateral compartments. Instead, intracellular radioactivity is slowly decreasing, providing a hint to intracellular degradation of the antibody.

In FIG. 5 it is shown that mAb 128.1 against the human transferrin receptor, unlike transferrin, co-localizes with late endosomal marker CD63 after internalization. hCMEC/D3 cells grown on collagen-coated coverslips were incubated with mAb 128.1 or FITC-labeled transferrin for ten minutes and then processed for immunofluorescence. mAb 128.1 was detected with Alexa-488-labeled secondary antibody (A), panel C shows transferrin-FITC fluorescence. Both preparations were counterstained with an antibody against the late endosomal marker CD63 and an Alexa-594-labeled secondary antibody (B, D). While mAb 128.1 shows a co-localization with CD63, transferrin is not found in the late-endosomal/lysosomal compartment, indicating re-routing of the transferrin receptor away from a recycling/transcytosis to a degradative trafficking pathway by mAb 128.1

In FIG. 6 it is shown that an antibody against human IGF-1 receptor (anti-IGF-1R antibody) is not transcytosed, but recycled to the extracellular medium. The transcytosis experiment was conducted as described above (see FIGS. 2 and 3) with the exception that antibody quantification was not done by radioactivity counting but by using a highly sensitive human IgG ELISA. It can be seen that the anti-IGF-1R antibody is not transcytosed, but recycled to the apical compartment, demonstrating that IGF-1 receptor is exclusively recycled in blood-brain barrier endothelial cells.

In FIG. 7 it is shown that mAb MEM-189 against the human transferrin receptor is, unlike mAb 128.1, recycled and transcytosed. The experiment was done as described above (see FIG. 6), using a mouse IgG ELISA for quantification. In contrast to mAb 128.1, which was not found in the apical or basolateral compartments (see above, FIG. 4), mAb MEM-189 is recycled and transcytosed to equal amounts, with a transfer rate slightly lower than that of transferrin (see also FIG. 3A).

In FIG. 8 it is shown that mAb MEM-189 binds in a pH-dependent fashion to the transferrin receptor, whereas mAb 128.1 does not show a pH-dependent binding. Binding of antibodies 128.1 and MEM-189 to the human transferrin receptor extracellular domain at pH 7.4 (extracellular pH) or pH 5.5 (endosomal pH) was measured by ELISA. While mAb 128.1 binds to the receptor at both pH values with similar affinity (triangles pH 7.4, crosses pH 5.5), mAb MEM-189 shows a strongly diminished binding at pH 5.5 (inverted triangles) as compared to pH 7.4 (circles). At pH 7.4, binding of mAb MEM-189 to the receptor is weaker than that of mAb 128.1 (see EC₅₀-values in the following Table). In the following table the EC₅₀ values for various antibodies against cell surface receptors and their respective EC₅₀ value ratios are shown.

TABLE

| | | | ratio |
|---|---|---|---|
| | | EC₅₀-value | EC_{50pH 5.5}/ |
| antigen | antibody/pH value | [ng/ml] | EC_{50pH 7.4} |
| transferrin | mAb 128.1/pH 5.5 | 9.8 | 1.3 |
| receptor | mAb 128.1/pH 7.4 | 7.8 | |
| transferrin | mAb MEM-189/pH 5.5 | >1000 | >15.6 |
| receptor | mAb MEM-189/pH 7.4 | 64.2 | |
| transferrin | mAb 13E4/pH 5.5 | 8.9 | 1.1 |
| receptor | mAb 13E4/pH 7.4 | 7.8 | |
| transferrin | mAb M-A712/pH 5.5 | 30 | 1.3 |
| receptor | mAb M_A712/pH 7.4 | 24 | |
| transferrin | mAb LT-71/pH 5.5 | 1174 | 1.8 |
| receptor | mAb LT-71/pH 7.4 | 663 | |
| transferrin | mAb MEM-75/pH 5.5 | 590 | 5.4 |
| receptor | mAb MEM-75/pH 7.4 | 110 | |
| transferrin | mAb OKT-9/pH 5.5 | 691 | 10.6 |
| receptor | mAb OKT-9/pH 7.4 | 65 | |
| INSR | mAb 83-14/pH 5.5 | 33.3 | 1.4 |
| | mAb 83-14/pH 7.4 | 23.1 | |
| INSR | mAb 243524/pH 5.5 | 275 | 1.4 |
| | mAb 243524/pH 7.4 | 203 | |
| transferrin | mAb 8D3/pH 5.5 | 54.9 | 1.1 |
| receptor | mAb 8D3/pH 7.4 | 51.1 | |
| transferrin | mAb R17217/pH 5.5 | 504 | 1.8 |
| receptor | mAb R17217/pH 7.4 | 285 | |

In FIG. 9 it is shown that mAbs 128.1 and MEM-189 compete for the same epitope on the transferrin receptor. The transferrin receptor extracellular domain was coated to a microtiter plate and pre-incubated with mAbs 128.1 or MEM-189, before the binding of the respective other mAb was detected. Binding of mAb MEM-189 to the receptor is fully blocked by mAb 128.1 pre-incubation (inverted triangles) as compared to binding in the absence of mAb 128.1 (circles). In contrast, binding of mAb 128.1 to the receptor is not inhibited by pre-incubation with mAb MEM-189 (triangles and crosses, respectively). In conclusion, mAb MEM-189 and mAb 128.1 compete for the same epitope on the human transferrin receptor. The fact that mAb MEM-189 cannot prevent binding of mAb 128.1 can be explained by the significantly higher affinity of mAb 128.1.

In FIG. 10 it is shown that antibodies M-A712 and 13E4 against the human transferrin receptor, both of which do not display pH-dependent binding, are not transcytosed through hCMEC/D3 cells.

The data presented above clearly demonstrate that the essential feature for antibody transcytosis is not the receptor epitope but the binding affinity to the receptor and the pH-dependent variation of the binding affinity.

### 1. Affinity

In certain embodiments, the binding site of the fusion polypeptide provided herein has a dissociation constant (Kd) of ≦10 μM, ≦1 μM, ≦100 μM, ≦10 nM, or ≦1 nM (e.g. in one embodiment of from about 10 ⁻⁵ M to about 10⁻⁹ M, or in another embodiment of about 10⁻⁷ M or less, e.g. from 10⁻⁷ M to 10⁻¹³ M, e.g., from 10⁻⁹ M to 10⁻¹³ M).

In one embodiment, Kd is measured by a radiolabeled antigen binding assay (RIA) performed wherein the binding site is a Fab fragment of an antibody and its antigen as described by the following assay. Solution binding affinity of FABs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen, Y., et al., J. Mol. Biol. 293 (1999) 865-881). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta, L. G., et al., Cancer Res. 57 (1997) 4593-4599). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE ®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μ/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen, Y., et al., J. Mol. Biol. 293 (1999) 865-881). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### 2. Antibody Fragments

In certain embodiments, a fusion polypeptide as reported herein comprises an antibody fragment as binding site. Antibody fragments include, but are not limited to, Fab, Fab′, Fab′-SH, F(ab′)₂, Fv, and scFv fragments, and other fragments described below. For a review of certain antibody fragments, see Hudson, P. J., et al., Nat. Med. 9 (2003) 129-134. For a review of scFv fragments, see, e.g., Plueckthun, A., In: The Pharmacology of Monoclonal Antibodies, Vol. 113, Rosenburg and Moore (eds.), Springer-Verlag, New York (1994), pp. 269-315; see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 0 404 097; WO 1993/01161; Hudson, P. J., et al., Nat. Med. 9 (2003) 129-134; and Holliger, P., et al., Proc. Natl. Acad. Sci. USA 90 (1993) 6444-6448. Triabodies and tetrabodies are also described in Hudson, P. J., et al., Nat. Med. 9 (2003) 129-134.

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Multispecific Antibodies

In certain embodiments, a fusion polypeptide as reported herein is a multispecific fusion polypeptide, e.g. a bispecific fusion polypeptide. Multispecific fusion polypeptides have binding specificities for at least two different sites. In certain embodiments, one of the binding specificities is for an internalizing cell surface receptor and the other is for a therapeutic target. In certain embodiments, a bispecific fusion polypeptide may bind to two different epitopes of the internalizing cell surface receptor. Bispecific fusion polypeptides can be prepared as full length fusion polypeptide or fusion polypeptide fragment.

In one embodiment the binding site of the fusion polypeptide is a complete antibody.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540), WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553); using “diabody” technology for making bispecific antibody fragments (see, e.g., Holliger, P., et al., Proc. Natl. Acad. Sci. USA 90 (1993) 6444-6448); and using single-chain Fv (sFv) dimers (see, e.g. Gruber, M., et al., J. Immunol., 152 (1994) 5368-5374); and preparing trispecific antibodies as described, e.g., in Tutt, A., et al., J. Immunol. 147 (1991) 60-69.

Engineered antibodies with three or more functional antigen binding sites, including “Octopus antibodies,” are also included herein (see, e.g. US 2006/0025576A1).

The antibody or fragment can be a “Dual Acting FAB” or “DAF” comprising an antigen binding site that binds to the internalizing cell surface receptor as well as another, different antigen (see, US 2008/0069820, for example).

The antibody or fragment herein also includes multispecific antibodies described in WO 2009/080251, WO 2009/080252, WO 2009/080253, WO 2009/080254, WO 2010/112193, WO 2010/115589, WO 2010/136172, PCT application No. PCT/EP2010/003559, or PCT application No. PCT/EP2010/003560.

### 4. Derivatives

In certain embodiments, a fusion polypeptide as reported herein may be further modified to contain additional non-proteinaceous moieties that are known in the art and readily available. The moieties suitable for derivatization of the fusion polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of a fusion polypeptide and non-proteinaceous moiety that may be selectively heated by exposure to radiation are provided. In one embodiment, the non-proteinaceous moiety is a carbon nanotube (Kam, N. W., et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

### 5. Assays

Fusion polypeptides as reported herein or components thereof may be identified, screened for, or characterized for their physical/chemical properties and/or biological activities by various assays known in the art.

### 5.1. Binding Assays and Other Assays

In one aspect, the binding site of the fusion polypeptide as reported herein is tested for its cell surface receptor binding activity, e.g., by known methods such as ELISA, Western blot, etc.

In another aspect, competition assays may be used to identify further binding sites, especially antibodies and antibody fragments that competes with mAb MEM-189 for binding to the transferrin receptor. In certain embodiments, such a competing antibody binds to the same epitope (e.g., a linear or a conformational epitope) that is bound by mAb MEM-189. Detailed exemplary methods for mapping an epitope to which an antibody binds are provided in Morris, G. E., (ed.), “Epitope Mapping Protocols,” In: Methods in Molecular Biology, Vol. 66, Humana Press, Totowa, N.J. (1996).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

For example, a fusion protein of the human transferrin receptor extracellular domain linked to human IgGl Fc can be coated to a 96-well plate by incubating 50 μl of a solution of 1 μg/ml in PBS for 1 h at RT. After 1 h of blocking with PBS/1% (w/v) BSA and four washes with PBS/0.1% (w/v) Tween, the antibody in question can be added to the plate at different concentrations in PBS/0.1% (w/v) BSA adjusted to pH 7.4 or pH 5.5 and incubated for 1.5 h at RT. After four washes with PBS/0.1% (w/v) Tween, bound antibodies can be detected using HRP-coupled secondary antibodies (30 min., RT) and 50 μl of TMB substrate. Color development can be stopped by addition of 50 μl of 1 N hydrochloric acid (HCl) and absorbance can be measured at 450 nm in a plate reader.

### 5.2. Activity Assays

Medium and supplements for hCMEC/D3 (see WO 2006/056879 and Weksler, B. B., et al., FASEB J. 19 (2005) 1872-1874) can be obtained from Lonza.

hCMEC/D3 cells (passages 26-29) can be cultured to confluence on collagen-coated coverslips (microscopy) or flasks in EBM2 medium containing 2.5% FBS, a quarter of the supplied growth factors and fully complemented with supplied hydrocortisone, gentamycin and ascorbic acid.

For all transcytosis assays, high density pore (1×10⁸pores/cm²) PET membrane filter inserts (0.4 μm pore size, 12 mm diameter) can be used in 12-well cell culture plates. Media volumes are calculated to be 400 μl and 1600 μl for apical and basolateral chambers, respectively. Apical chambers of filter inserts can be coated with rat tail collagen I (7.5 μg/cm²) followed by fibronectin (5 μg/ml), each incubation lasting for 1 h at RT. hCMEC/D3 cells can be grown to confluent monolayers (˜2×10⁵ cells/cm²) for 10-12 days in EBM2 medium. Empty filters can be blocked in PBS containing 1% BSA for 1 h or overnight (o/n) before assay and then calibrated for at least 1 h in EBM2 before the assay.

The assay (see FIG. 2 for assay scheme) can be performed in serum-free EBM2 medium which was otherwise reconstituted as described herein. On day of the assay, cells are serum-starved for 60 min. to deplete the natural ligand of the internalizing cell surface receptor in question. Filter inserts with or without (but blocked overnight in complete medium) cells were incubated apically with radiolabeled natural ligand of the internalizing cell surface receptor, ¹²⁵I-labeled or unlabeled monoclonal antibodies in question for 1 h at 37° C. Afterwards the entire apical and basolateral volume are collected. Paracellular flux can be calculated from the determined values. The monolayers were washed at RT in serum-free medium apically (400 μl) and basolaterally (1600 μl) three times for 3-5 min. each. All wash volumes were collected to monitor efficiency of removal of the unbound ligand or antibody. Pre-warmed medium was added to the apical chamber and the filters transferred to a fresh 12 well plate (blocked overnight with PBS containing 1% BSA) containing 1600 μl pre-warmed medium. At this point, filters with or without cells were lysed in 500 μl RIPA buffer in order to determine specific ligand or antibody uptake. The remaining filters were incubated at 37° C. or at 4° C. and samples collected at various time points to determine apical and/or basolateral release of ligand or antibody. Intact and degraded ¹²⁵I-labeled natural ligand or ¹²⁵I-labeled antibody was assessed using trichloro acetic acid (TCA) precipitation. The quantity of radioactive natural ligand or antibody in supernatants or lysates can be determined by gamma-radiation counting. The content of unlabeled antibody in the samples can be quantified using a highly sensitive IgG ELISA (see Example 3). For each time point, data should be generated from two empty filters and three filter cell cultures.

### 6. Recombinant Methods and Compositions

Fusion polypeptides may be produced using recombinant methods and compositions. In one embodiment, isolated nucleic acid encoding a fusion polypeptide as reported herein is provided. In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with) one or more vector comprising a nucleic acid that encodes an amino acid sequence comprising the fusion polypeptide. In one embodiment, the host cell is eukaryotic, e.g. a Chinese Hamster Ovary (CHO) cell or lymphoid cell (e.g., Y0, NS0, SP2/0 cell). In one embodiment, a method of making a fusion polypeptide as reported herein is provided, wherein the method comprises culturing a host cell comprising a nucleic acid encoding the fusion polypeptide, as provided above, under conditions suitable for expression of the fusion polypeptide, and optionally recovering the fusion polypeptide from the host cell (or host cell culture medium).

For recombinant production of a fusion polypeptide as reported herein, nucleic acid encoding a fusion polypeptide as reported herein, e.g., as described above, is isolated and inserted into one or more vectors for further cloning and/or expression in a host cell. Such nucleic acid may be readily isolated and sequenced using conventional procedures.

Suitable host cells for cloning or expression of polypeptide-encoding vectors include prokaryotic or eukaryotic cells described herein. For example, polypeptide may be produced in bacteria, in particular when glycosylation is not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C., (ed.), Humana Press, Totowa, N.J. (2003), pp. 245-254, describing expression of antibody fragments in E. coli.) After expression, the polypeptide may be isolated from the bacterial cell paste in a soluble fraction and can be further purified.

In addition to prokaryotes, eukaryotic microbes such as filamentous fungi or yeast are suitable cloning or expression hosts for polypeptide-encoding vectors, including fungi and yeast strains whose glycosylation pathways have been “humanized,” resulting in the production of a fusion polypeptide with a partially or fully human glycosylation pattern. See Gerngross, T. U., Nat. Biotech. 22 (2004) 1409-1414, and Li, H., et al., Nat. Biotech. 24 (2006) 210-215.

Suitable host cells for the expression of glycosylated polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L., et al., J. Gen. Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P., et al., Annals N.Y. Acad. Sci. 383 (1982) 44-68; MRC 5 cells; and FS4 cells. Other useful mammalian host cell lines include Chinese hamster ovary (CHO) cells, including DHFR⁻ CHO cells (Urlaub, G., et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., In: Methods in Molecular Biology, Vol. 248, Lo, B. K. C. (ed.), Humana Press, Totowa, N.J. (2004) pp. 255-268.

### 7. Immunoconjugates

Herein are also provided fusion polypeptides in which at least one of the components such as the effector moiety is e.g. a cytotoxic agent, such as a chemotherapeutic agent or drug, a growth inhibitory agent, a toxin (e.g., a protein toxin, an enzymatically active toxin of bacterial, fungal, plant, or animal origin, or fragments thereof), or a radioactive isotope.

In one embodiment the effector moiety is a drug or a pharmaceutically active compound, including but not limited to a maytansinoid (see U.S. Pat. No. 5,208,020, U.S. Pat. No. 5,416,064, EP 0 425 235), an auristatin such as monomethyl auristatin drug moieties DE and DF (MMAE and MMAF, see U.S. Pat. No. 5,635,483, U.S. Pat. No. 5,780,588, U.S. Pat. No. 7,498,298), a dolastatin, a calicheamicin or derivative thereof (see U.S. Pat. No. 5,712,374, U.S. Pat. No. 5,714,586, U.S. Pat. No. 5,739,116, U.S. Pat. No. 5,767,285, U.S. Pat. No. 5,770,701, U.S. Pat. No. 5,770,710, U.S. Pat. No. 5,773,001, U.S. Pat. No. 5,877,296, Hinman, L. M., et al., Cancer Res. 53 (1993) 3336-3342, Lode, H. N., et al., Cancer Res. 58 (1998) 2925-2928), an anthracycline such as daunomycin or doxorubicin (see Kratz, F., et al., Current Med. Chem. 13 (2006) 477-523, Jeffrey, S. C., et al., Bioorg. Med. Chem. Letters 16 (2006) 358-362, Torgov, M. Y., et al., Bioconjug. Chem. 16 (2005) 717-721, Nagy, A., et al., Proc. Natl. Acad. Sci. (USA) 97 (2000) 829-834, Dubowchik, G. M., et al., Bioorg. Med. Chem. Lett. 12 (2002) 1529-1532, King, H. D., et al., J. Med. Chem. 45 (2002) 4336-4343, and U.S. Pat. No. 6,630,579), methotrexate, vindesine, a taxane such as docetaxel, paclitaxel, larotaxel, tesetaxel, and ortataxel, a trichothecene, and CC 1065.

In another embodiment the effector moiety is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment the effector moiety is a radioactive atom. A variety of radioactive isotopes are available for the production of radioconjugates. Examples include At²¹¹, I¹³¹, I₁₂₅, Y⁹⁰, Re¹⁸⁶, Re¹⁸⁸, Sm¹⁵³, Bi²¹², P³²Pb²¹², and radioactive isotopes of Lu. When the radioconjugate is used for detection, it may comprise a radioactive atom for scintigraphic studies, for example Tc⁹⁹m or I¹²³, or a spin label for nuclear magnetic resonance (NMR) imaging (also known as magnetic resonance imaging, MRI) I¹²³ again, such as I¹³¹, In¹¹¹, F¹⁹, C¹³, N¹⁵, O¹⁷, gadolinium, manganese or iron.

The effector moiety can be fused to the binding site in the fusion polypeptide as reported herein using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido components (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine components (such as 1,5-difluoro-2,4-dinitrobenzene). For example, a ricin immunotoxin can be prepared as described in Vitetta, E. S., et al., Science 238 (1987) 1098-1104. Carbon-14-labeled 1-isothiocyanatobenzyl-3-methyldiethylene triamine pentaacetic acid (MX-DTPA) is an exemplary chelating agent for conjugation of radionucleotide to the fusion polypeptide (see WO 94/11026). The linker for conjugating the toxic moiety to the fusion polypeptide as reported herein can be a “cleavable linker” facilitating release of a cytotoxic drug in the cell. For example, an acid-labile linker, peptidase-sensitive linker, photolabile linker, dimethyl linker, or disulfide-containing linker (Chari, R. V., et al., Cancer Res. 52 (1992) 127-131, U.S. Pat. No. 5,208,020) can be used.

The effector moiety can be fused to the binding site in the fusion polypeptide as reported herein via a linker, which is e.g. but not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., USA).

The effector moiety can be fused via a peptidic linker to the binding site. In one embodiment the peptidic linker has from 4 to 20 amino acid residues. In one embodiment the linker is the same between the repeat-motif-molecules, in another embodiment the conjugate contains linker with two or more different amino acid sequences. In a further embodiment the linker is selected from (G₃S), (G₃S)₂, (G₃S)₃, (G₃S)₄, (G₃S)₅, (G₄S), (G₄S)₂, (G₄S)₃, (G₄S)₄, (G₄S)₅ (SEQ ID NO: 1 and SEQ ID NO: 12 to 20), especially from (G₄S)₃ and (G₄S)₄ (SEQ ID NO: 18 and SEQ ID NO: 19).

### 8. Methods and Compositions for Diagnostics and Detection

In certain embodiments any of the fusion polypeptides provided herein is useful for detecting the presence of a target specifically bound by a binding site in the fusion polypeptide in a biological sample. The term “detecting” as used herein encompasses quantitative or qualitative detection.

In one embodiment the fusion polypeptide is provided for use in a method of diagnosis or detection. In a further aspect, a method of detecting the presence of the target of the binding site or the effector moiety of the fusion polypeptide as reported herein in a biological sample is provided. In certain embodiments, the method comprises contacting the biological sample with a fusion polypeptide as reported herein under conditions permissive for binding of the binding site(s) or the effector moiety to its target(s), and detecting whether a complex is formed between the fusion polypeptide and the target. Such method may be an in vitro or in vivo method. In one embodiment the fusion polypeptide as reported herein is used to select subjects eligible for therapy with an isolated polypeptide comprised in the fusion polypeptide, e.g. where the target is a biomarker for selection of patients.

In certain embodiments a labeled fusion polypeptide is provided, i.e. a fusion polypeptide wherein the effector moiety is a label. Labels include, but are not limited to, labels that are detected directly (such as fluorescent, chromophoric, electron-dense, chemiluminescent, and radioactive labels), as well as labels, such as enzymes or ligands, that are detected indirectly, e.g., through an enzymatic reaction or molecular interaction. Exemplary labels include, but are not limited to, the radioisotopes P³², C¹⁴, I¹²⁵, H³, and I¹³¹, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### III. Pharmaceutical Formulations

Pharmaceutical formulations of a fusion polypeptide as reported herein are prepared by mixing such fusion polypeptide having the desired degree of purity with one or more optional pharmaceutically acceptable carriers (Osol, A., (ed.) Remington's Pharmaceutical Sciences 16th edition (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyl dimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight (less than about 10 residues) polypeptides, proteins, such as serum albumin, gelatin, or immunoglobulins, hydrophilic polymers such as polyvinyl pyrrolidone, amino acids such as glycine, glutamine, asparagine, histidine, arginine, or lysine, monosaccharides, disaccharides, and other carbohydrates including glucose, mannose, or dextrins, chelating agents such as EDTA, sugars such as sucrose, mannitol, trehalose or sorbitol, salt-forming counter-ions such as sodium, metal complexes (e.g. Zn-protein complexes), and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rhuPH2O (HYLENEX®, Baxter International, Inc.). Certain exemplary sHASEGPs and methods of use, including rhuPH2O, are described in US 2005/0260186 and US 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, especially those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Osol, A., (ed.) Remington's Pharmaceutical Sciences 16th edition (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### IV. Therapeutic Methods and Compositions

Any of the fusion polypeptides as reported herein wherein the effector moiety is a therapeutically active compound or a detectable label may be used in therapeutic methods.

In one aspect a fusion polypeptide as reported herein for use as a medicament is provided. In further aspects a fusion polypeptide for use in treating CNS-related disease is provided. In certain embodiments a fusion polypeptide for use in a method of treatment is provided. In certain embodiments the invention provides a fusion polypeptide for use in a method of treating an individual having a CNS-related disease comprising administering to the individual an effective amount of the fusion polypeptide. In one such embodiment the method further comprises administering to the individual an effective amount of at least one additional therapeutic agent. In further embodiments the invention provides a fusion polypeptide as reported herein for use in reversing or stabilizing of a CNS-related disease. In certain embodiments, the invention provides a fusion polypeptide for use in reversing or stabilizing of a CNS-related disease in an individual comprising administering to the individual an effective amount of the fusion polypeptide to reverse or stabilize a CNS-related disease. An “individual” according to any of the above embodiments is especially a human.

CNS-related diseases include e.g. viral or bacterial diseases (such as encephalitis, meningitis), cancer (such as brain cancer), neurodegenerative diseases (such as Alzheimer's disease, Parkinson's disease, Huntington's disease, amyotrophic lateral sclerosis (ALS, Lou Gehrig's disease), multiple sclerosis), acute diseases (such as stroke, physical trauma, spinal cord injury), psychiatric diseases (such as anxiety, depression, epilepsies, seizure disorders, schizophrenia, sleep disorders), cognition diseases (such as memory diseases, cognition diseases), cerebrovascular disorders (ischemic stroke, intracerebral hemorrhage, subarachnoid hemorrhage), pain-related diseases, prion diseases (such as Creutzfeldt Jakob disease, bovine spongiform encephalopathy), or addiction diseases (such as alcoholism).

The fusion polypeptide as reported herein is therapeutically effective if it resulted in the reversal or stabilization of a CNS-related disease.

In one embodiment the effector moiety is a therapeutically active compound which is binding to or modifies the activity of brain derived neurotropic factor (BDNF), ciliary neurotropic factor (CNTF), glial cell-line neurotropic factor (GDNF), insulin-like growth factor (IGF), or nerve growth factor (NGF).

In one embodiment the effector moiety is a therapeutically active compound selected from cholecystokinin (CCK), dopamine, an endorphin, an encephalin, gamma-amino-butyric acid (GABA), neuropeptide Y, substance P, thyrotropin releasing hormone (TRH), or vasoactive intestinal peptide (VIP).

In one embodiment the effector moiety is a therapeutically active compound selected from an anticonvulsant, an anxiolytic agent, a cytokine, or a polynucleotide such as siRNA.

In a further aspect as reported herein is provided the use of a fusion polypeptide as reported herein in the manufacture or preparation of a medicament. In one embodiment the medicament is for treatment of a CNS-related disease. In a further embodiment the medicament is for use in a method of treating a CNS-related disease comprising administering to an individual having a CNS-related disease an effective amount of the medicament. In a further embodiment the medicament is for reversing or stabilizing a CNS-related disease. In a further embodiment the medicament is for use in a method of reversing or stabilizing a CNS-related disease in an individual comprising administering to the individual an amount of the medicament effective to reverse or stabilize a CNS-related disease. An “individual” according to any of the above embodiments may be a human.

In a further aspect as reported herein a method for treating a CNS-related disease is provided. In one embodiment the method comprises administering to an individual having such a disease an effective amount of a fusion polypeptide as reported herein. An “individual” according to any of the above embodiments may be a human.

In a further aspect as reported herein a method for reversing or stabilizing a CNS-related disease in an individual is provided. In one embodiment the method comprises administering to the individual an effective amount of a fusion polypeptide as reported herein to reverse or stabilize a CNS-related disease. In one embodiment an “individual” is a human.

In a further aspect as reported herein a pharmaceutical formulation comprising any one of the fusion polypeptides provided herein, e.g., for use in any of the above therapeutic methods is provided. In one embodiment the pharmaceutical formulation comprises any one of the fusion polypeptides provided herein and a pharmaceutically acceptable carrier. In another embodiment the pharmaceutical formulation comprises any one of the fusion polypeptides as reported herein and at least one additional therapeutic agent.

Fusion polypeptides as reported herein can be used either alone or in combination with other agents in a therapy. For instance, a fusion polypeptide as reported herein may be co-administered with at least one additional therapeutic agent.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant. Fusion polypeptides as reported herein can also be used in combination with radiation therapy.

A fusion polypeptide as reported herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Fusion polypeptides as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The fusion polypeptide need not be, but is optionally formulated with one or more agents currently used to prevent or treat the disorder in question. The effective amount of such other agents depends on the amount of fusion polypeptide present in the formulation, the type of disorder or treatment, and other factors discussed above. These are generally used in the same dosages and with administration routes as described herein, or about from 1% to 99% of the dosages described herein, or in any dosage and by any route that is empirically/clinically determined to be appropriate.

For the prevention or treatment of disease, the appropriate dosage of a fusion polypeptide as reported herein (when used alone or in combination with one or more other additional therapeutic agents) will depend on the type of disease to be treated, the type of fusion polypeptide, the severity and course of the disease, whether the fusion polypeptide is administered for preventive or therapeutic purposes, previous therapy, the patient's clinical history and response to the fusion polypeptide, and the discretion of the attending physician. The fusion polypeptide is suitably administered to the patient at one time or over a series of treatments. Depending on the type and severity of the disease, about 1 μg/kg to 15 mg/kg (e.g. 0.1 mg/kg-10 mg/kg) of fusion polypeptide can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion polypeptide would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion polypeptide). An initial higher loading dose, followed by one or more lower doses may be administered. The progress of this therapy can be easily monitored by conventional techniques and assays.

### Articles of Manufacture

In another aspect as reported herein an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a fusion polypeptide as reported herein. The label or package insert indicates that the composition is used for treating the condition of choice. Moreover, the article of manufacture may comprise (a) a first container with a composition contained therein, wherein the composition comprises a fusion polypeptide as reported herein; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following examples, sequence listing and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Schematic drawing of the pH-dependent transcytosis mechanism.

FIG. 2 Set-up of hCMEC/D3 transcytosis assay.

FIG. 3 Assay validation by transcytosis of ¹²⁵I-transferrin through hCMEC/D3 brain endothelial cells.

FIG. 4 mAb 128.1 against the human transferrin receptor does not leave hCMEC/D3 cells.

FIG. 5 mAb 128.1 against the human transferrin receptor, unlike transferrin, co-localizes with late endosomal marker CD63 after internalization.

FIG. 6 anti-IGF-1R antibody against the human IGF-1 receptor is not transcytosed, but recycled.

FIG. 7 mAb MEM-189 against the human transferrin receptor is, unlike mAb 128.1, recycled and transcytosed.

FIG. 8 mAb MEM-189 binds in a pH-dependent fashion to the transferrin receptor, mAb 128.1 does not.

FIG. 9 mAbs 128.1 and MEM-189 compete for the same epitope.

FIG. 10 mAbs 13E4 and M-A712 against the human transferrin receptor are not transcytosed.

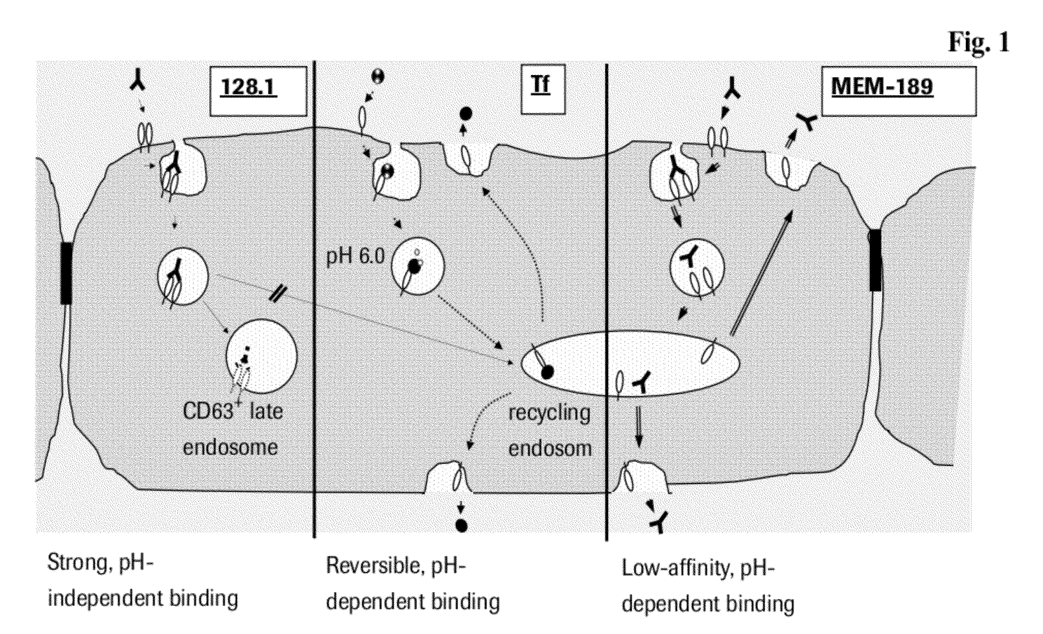

### DESCRIPTION OF THE SEQUENCES

SEQ ID NO: 01 to 20 linker polypeptide amino acid sequences
SEQ ID NO: 21 mAb 128.1, heavy chain variable domain amino acid sequence
SEQ ID NO: 22 mAb 128.1, variable domain light chain amino acid sequence
SEQ ID NO: 23 human IgG1 constant region amino acid sequence
SEQ ID NO: 24 human IgG4 constant region amino acid sequence
SEQ ID NO: 25 human kappa light chain constant domain amino acid sequence
SEQ ID NO: 26 human lambda light chain constant domain amino acid sequence

### EXAMPLES

### Example 1

### hCMEC/D3 Cell Culture for Transcytosis Assays or Fluorescence Microscopy

Medium and supplements for hCMEC/D3 (see WO 2006/056879 and Weksler, B. B., et al., FASEB J. 19 (2005) 1872-1874) were obtained from Lonza. hCMEC/D3 cells (passages 26-29) were cultured to confluence on collagen-coated coverslips (microscopy) or flasks in EBM2 medium containing 2.5% FBS, a quarter of the supplied growth factors and fully complemented with supplied hydrocortisone, gentamycin and ascorbic acid.

For all transcytosis assays, high density pore (1×10⁸pores/cm²) PET membrane filter inserts (0.4 μm pore size, 12 mm diameter) were used in 12-well cell culture plates. Media volumes were calculated to be 400 μl and 1600 μl for apical and basolateral chambers, respectively. Apical chambers of filter inserts were coated with rat tail collagen I (7.5 μcm²) followed by fibronectin (5 μg/ml), each incubation lasting for 1 h at RT. hCMEC/D3 cells were grown to confluent monolayers (˜2×10⁵ cells/cm²) for 10-12 days in EBM2 medium. Empty filters were blocked in PBS containing 1% BSA for 1 h or overnight (o/n) before assay and then calibrated for at least 1 h in EBM2 medium before the assay.

### Example 2

### Transcytosis Assay of ¹²⁵I-transferrin and Monoclonal Antibodies

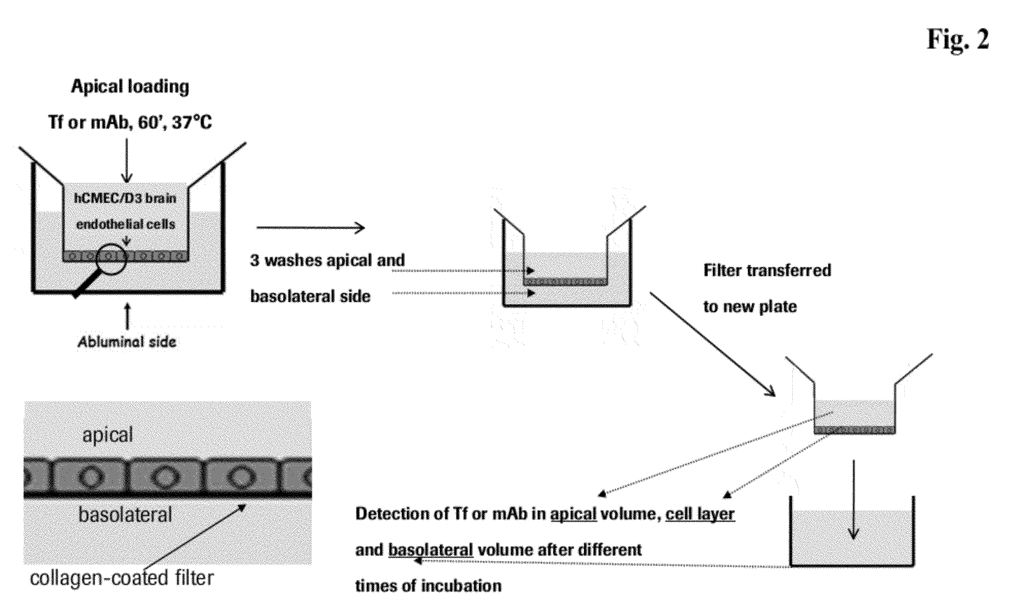

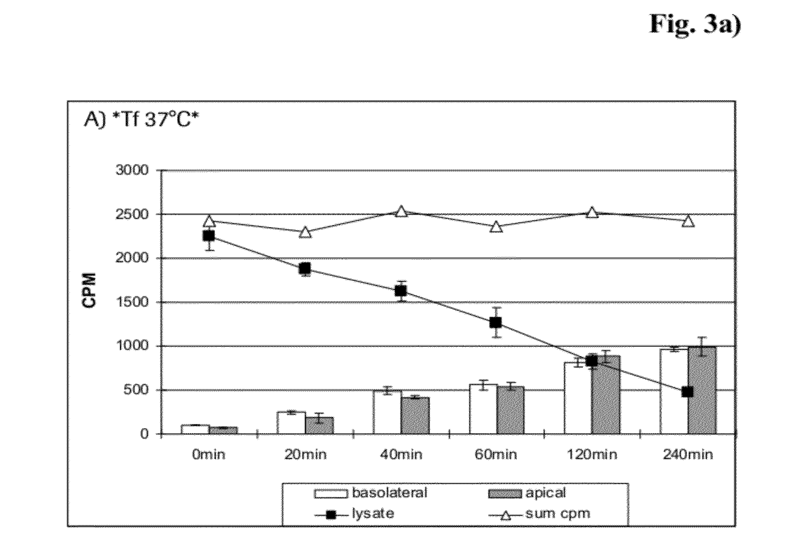

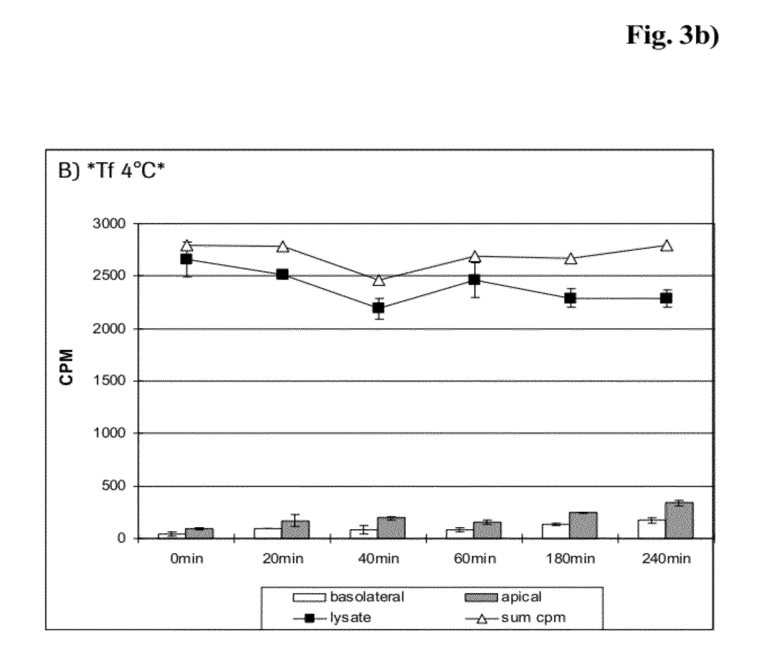

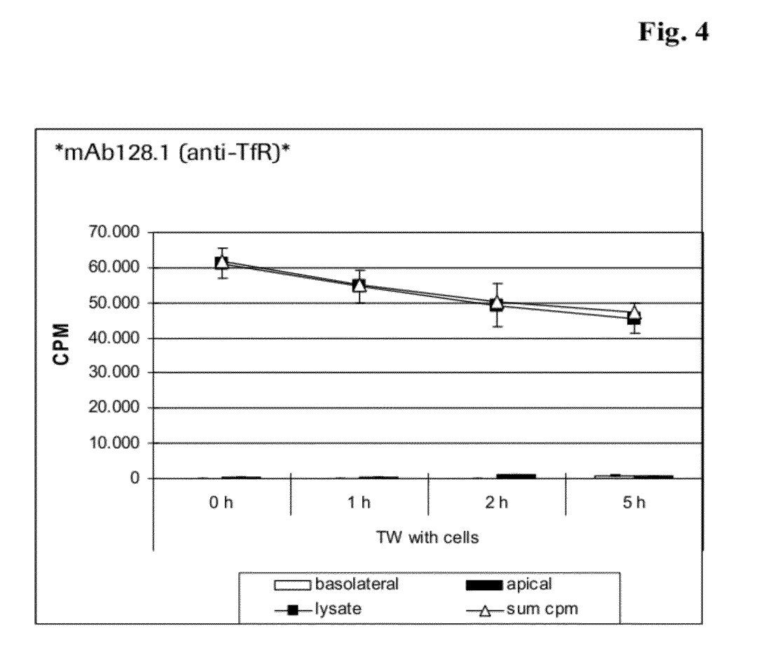

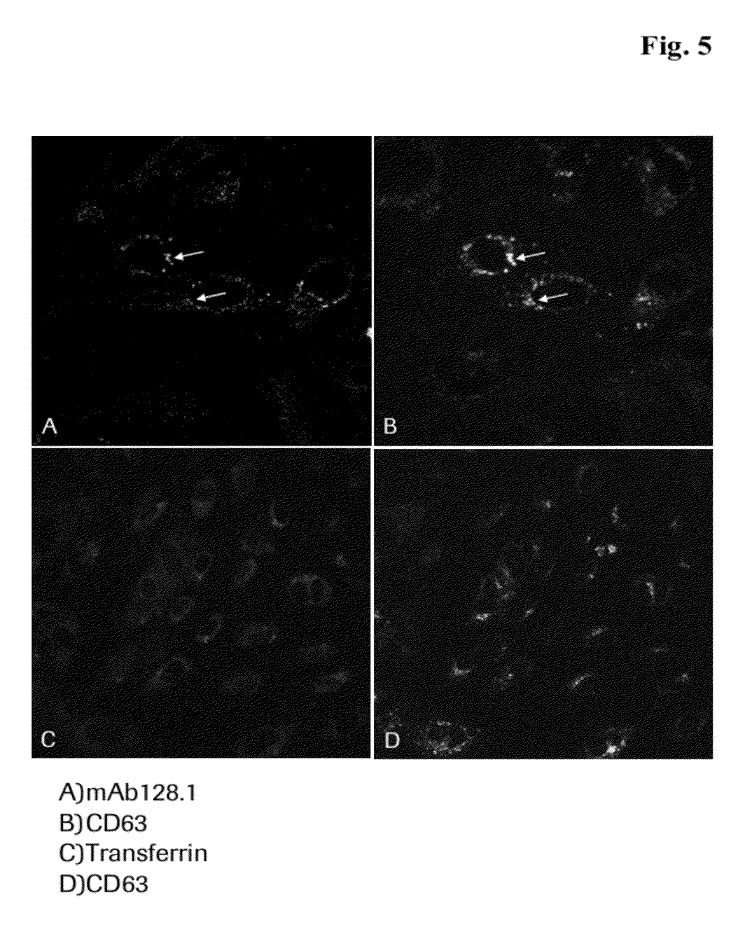

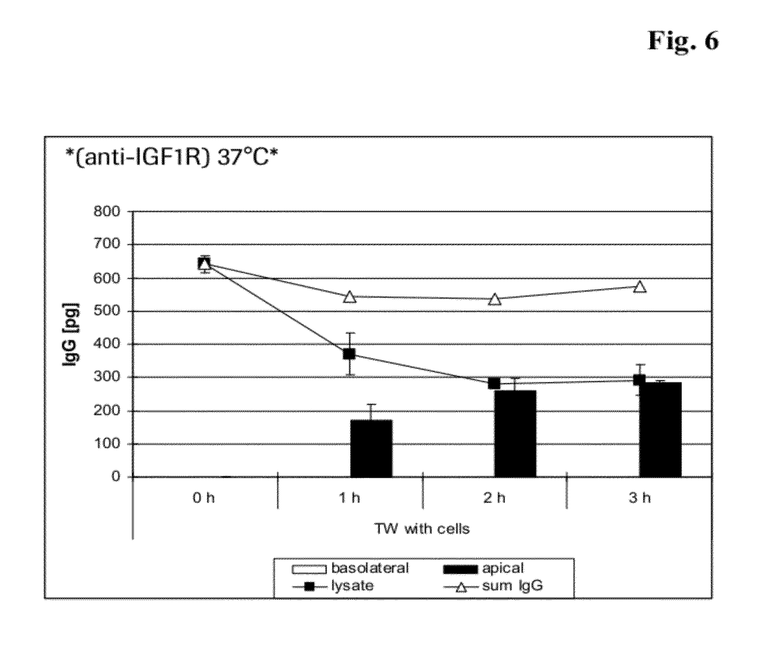

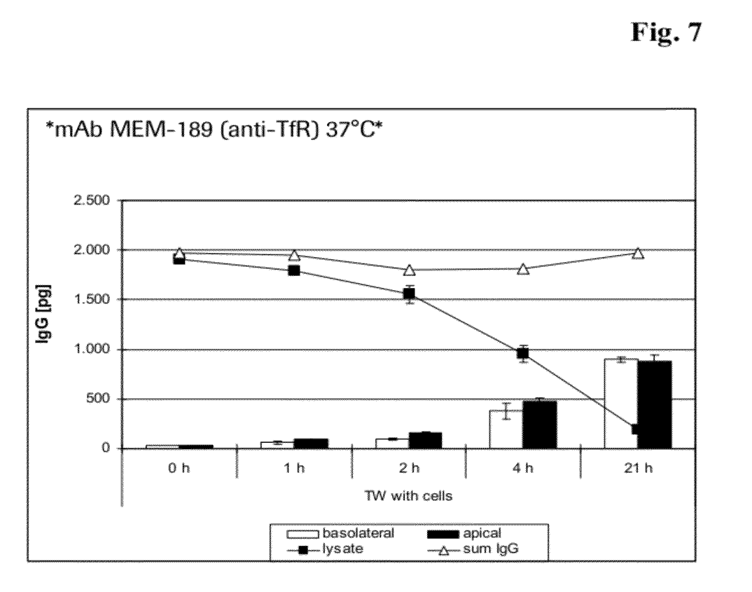

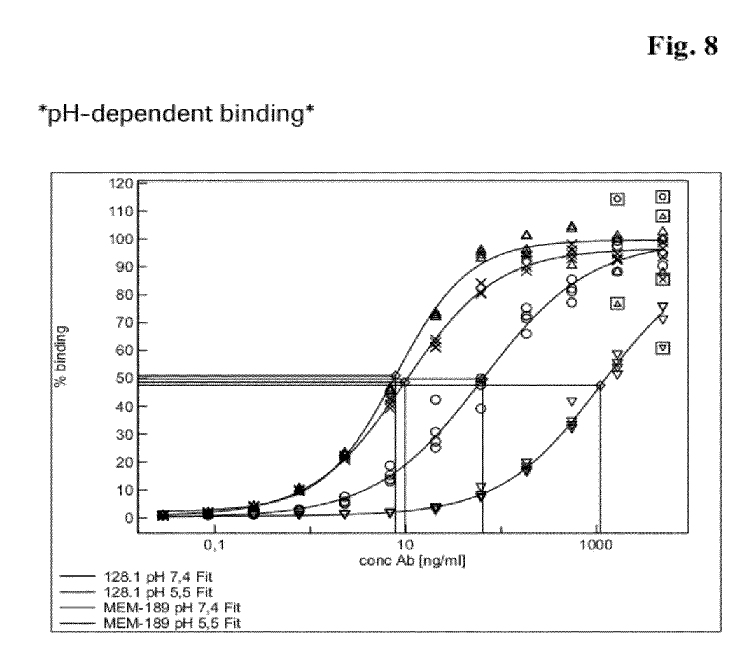

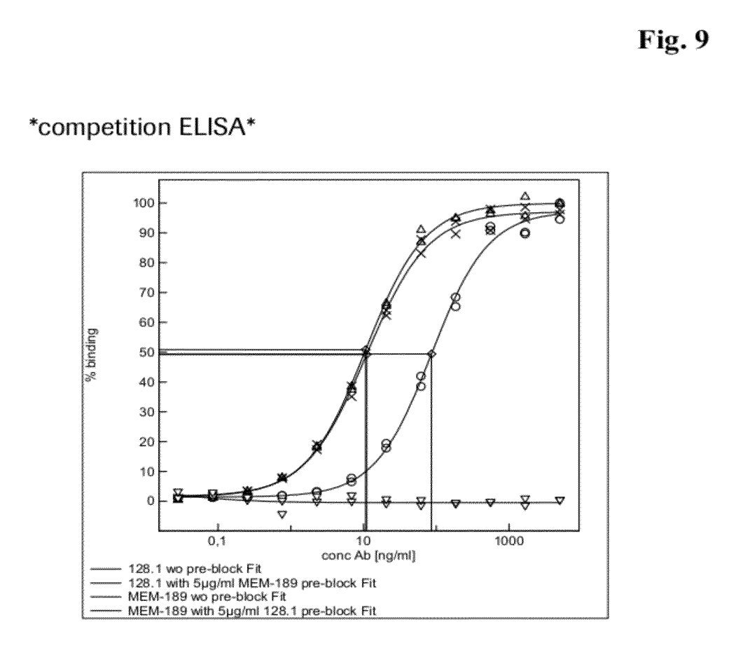

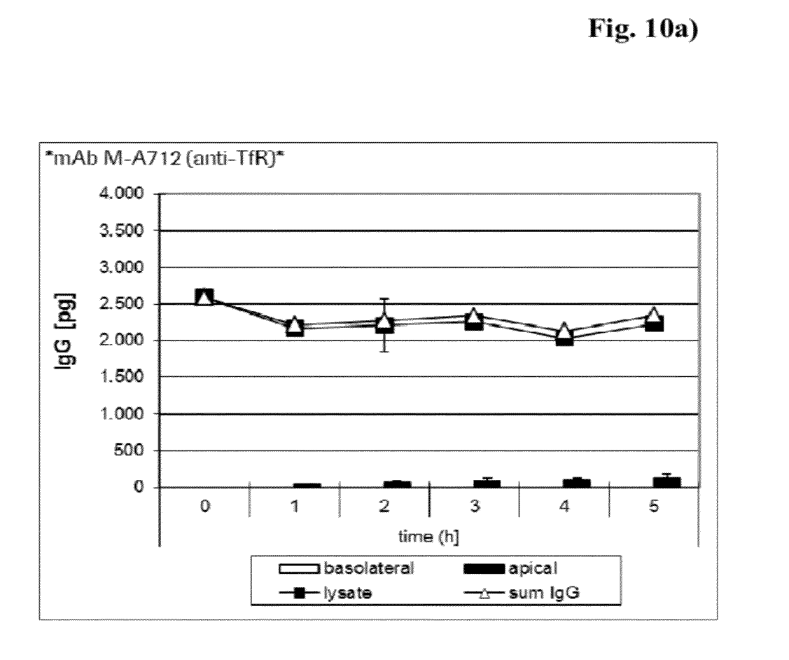

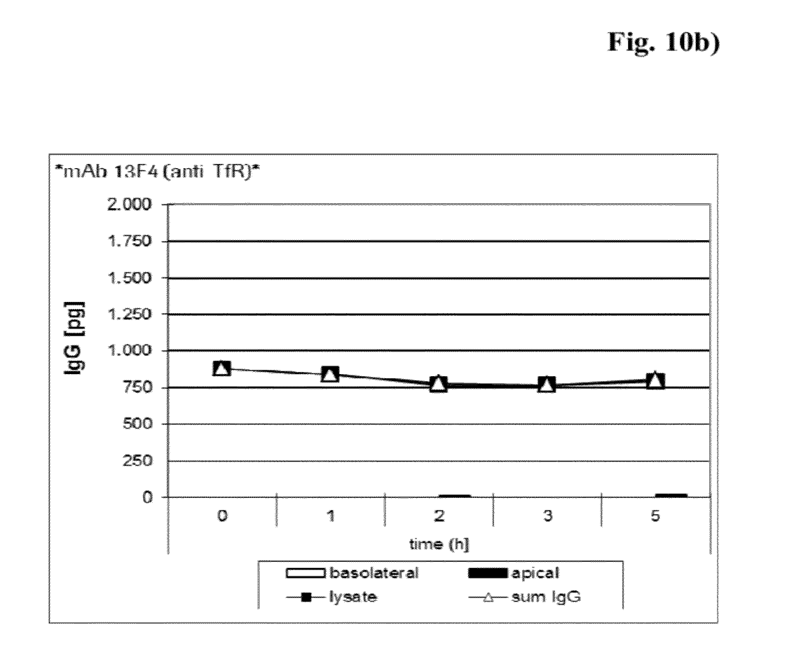

¹²⁵I-transferrin (Tfn) was obtained from Perkin Elmer (Perkin Elmer, Rodgau, Germany, #NEX212050UC). mAb 128.1 against the human and mAb 8D3 against the mouse transferrin receptor were transiently expressed in HEK cells transfected with a vector comprising a continuous open reading frame of the coding sequences of human IgG1 heavy and light chain constant regions, respectively, and the variable regions of the mouse anti-human transferrin-receptor antibody 128.1 (for variable region sequences see WO 93/10819 and SEQ ID NO: 21 and 22) or the rat anti-mouse transferrin-receptor antibody 8D3 (Boado et al. (2009), Biotechnol. Bioeng. 102, 1251-1258) and purified as reported previously. mAb 128.1 was also labeled with ¹²⁵I. A monoclonal antibody against the human IGF-1 receptor was expressed and purified as described in U.S. Pat. No. 7,572,897. Mouse monoclonal mAbs MEM-189 and 13E4 against the human transferrin receptor were obtained from Abcam (Cambridge, England, #ab1086 and #ab38171, respectively) and mAb M-A712 from BD Biosciences (Heidelberg, Germany, #555534). The entire assay (see FIG. 2 for assay scheme) was performed in serum-free EBM2 medium which was otherwise reconstituted as described in Example 1. On day of the assay, cells were serum-starved for 60 min. to deplete transferrin (only for transferrin transcytosis). Filter inserts with or without (but blocked overnight in complete medium) cells were incubated apically with radiolabeled transferrin, ¹²⁵I-labeled or unlabeled monoclonal antibodies for 1 h at 37° C. Afterwards the entire apical and basolateral volume were collected. Paracellular flux and stability of the radio-iodinated ligand were calculated from the determined values. The monolayers were washed at RT in serum-free medium apically (400 μl) and basolaterally (1600 μl) three times for 3-5 min. each. All wash volumes were collected to monitor efficiency of removal of the unbound ligand or antibody. Pre-warmed medium was added to the apical chamber and the filters transferred to a fresh 12 well plate (blocked overnight with PBS containing 1% BSA) containing 1600 μl pre-warmed medium. At this point, filters with or without cells were lysed in 500 μl RIPA buffer (Sigma, Munich, Germany, #R0278) in order to determine specific ligand or antibody uptake. The remaining filters were incubated at 37° C. or at 4° C. and samples collected at various time points to determine apical and/or basolateral release of ligand or antibody. Intact and degraded ¹²⁵I-transferrin or ¹²⁵I-mAb 128.1 was assessed using trichloro acetic acid (TCA) precipitation. The quantity of radioactive transferrin or mAb 128.1 in supernatants or lysates was determined by gamma-radiation counting. The content of unlabeled antibody in the samples was quantified using a highly sensitive IgG ELISA (see Example 3). For each time point, data was generated from two empty filters and three filter cell cultures.

### Example 3

### Sensitive IgG ELISA after Transcytosis Assay

The entire procedure was performed at RT using an automated washer for the wash steps. A 384-well plate was coated with 30 μl/well of 1 μg/ml anti-human/mouse-IgG, Fcγ-specific (Dianova, Hamburg, Germany, #109-005-098 or #115-005-164, respectively) in phosphate buffered saline solution (PBS) for 2 h followed by 1 h incubation in blocking buffer PBS containing 1% (w/v) BSA (Sigma, Munich, Germany, #A2153) for human and mouse IgG assays, respectively. Serially diluted samples from the transcytosis assay and standard concentrations of the antibody used in the transcytosis assay were added to the plate and incubated for 2 h. After four washes, 30 μl/well of 50 ng/ml anti-human/mouse-F(ab)₂-biotin-conjugate (Dianova, Hamburg, Germany, #109-066-097 or #115-066-072, respectively) in blocking buffer (see above) was added and incubated for a further 2 h. Following six washes, 30 μl/well of 50 ng/ml (huIgG assay) or 100 ng/ml (mIgG assay) poly-HRP40-streptavidin (Fitzgerald, Acton (MA), USA, #65R-S104PHRPx; in PBS containing 1% (w/v) BSA and 0.05% (w/v) Tween-20) was added and incubated for 30 min. After four washes, immune complexes were detected by addition of 30 μl/well of BM Chemiluminescence Substrate (Roche Diagnostics GmbH, Mannheim, Germany). The luminescence signal was measured using a luminescence plate reader and concentration calculated using the fitted standard curve. The range of the assay was from 10 pg/ml to 10 ng/ml.

### Example 4

### Confocal Fluorescence Microscopy

To investigate the localization of the 128.1 antibody and holo-transferrin, monolayers of hCMEC/D3 cells grown to confluence on collagen-coated coverslips were incubated with 5 μg/ml FITC-tagged holo-transferrin (Invitrogen, Darmstadt, Germany, #T-2871) or 1 μg/ml mAb 128.1 for 10 min. Thereafter the medium was removed and replaced with fresh medium. After 1 h at 37° C., the monolayers were fixed in 4% paraformaldehyde (PFA) for 15 min. at RT, permeabilized for 10 min. (PBS/0.1% (w/v) Triton X-100 (Sigma, Munich, Germany, #93443) and incubated with an antibody against late endosomal/lysosomal marker CD63 (R&D Systems, Wiesbaden, Germany, #MAB5417) for 45 min. at RT. Cells were subjected to washes in PBS/0.1% (w/v) Triton X-100 for 15 min. and sequentially incubated, where necessary, with secondary antibodies (goat anti-human IgG-Alexa Fluor 488 and/or chicken anti-mouse IgG-Alexa Fluor 594 (Invitrogen, Darmstadt, Germany, #A11013 or #A21201, respectively) for 45 min. at RT. Cells were washed in PBS/0.1% (w/v) Triton X-100 for 30 min. Afterwards cover slips were mounted in mounting medium. Fluorescent images were obtained using a confocal microscope. All the confocal images show a single, representative, section of a Z-series taken through the entire cell.

### Example 5

### pH-dependent Binding and Competition ELISA

A fusion protein of the human transferrin receptor extracellular domain linked to human IgG1 Fc (R&D Systems, Wiesbaden, Germany, # 2474-TR-050) or the extracellular domain of the mouse transferrin receptor (SinoBiological, Beijing, China, #50741-MO7H) or of the human insulin receptor (R&D Systems, Wiesbaden, Germany, #1544-IR/CF) were coated to a 96-well plate by incubating 50 μl of a solution of 1 μg/ml in PBS for 1 h at RT. After 1 h of blocking with PBS/1% (w/v) BSA and four washes with PBS/0.1% (w/v) TWEEN 20, antibodies MEM-189, 128.1, 13E4, M-A712, LT-71, MEM-75 (both Abcam, #ab9179 and #ab38446, respectively), OKT-9 (eBioscience, Frankfurt, Germany, #16-0719; all against the human TfR), 8D3, R17217 (Santa Cruz, Heidelberg, Germany, #sc-52504; both against the mouse TfR), 83-13 (Invitrogen, Darmstadt, Germany, #AHR0221) and 243524 (R&D Systems, #MAB1544; both against the human insulin receptor) were added to the plate at different concentrations in PBS/0.1% (w/v) BSA adjusted to pH 7.4 or pH 5.5 and incubated for 1.5 h at RT. Alternatively, wells were incubated with mAb MEM-189 or mAb 128.1 as blocking antibodies at a fixed concentration of 5 μg/ml, washed four times, and afterwards incubated for 30 min. at RT with different concentrations of the other antibody that had not been used for blocking After four further washes with PBS/0.1% (w/v) TWEEN 20, bound antibodies were detected using HRP-coupled secondary antibodies (Dianova, Hamburg, Germany, #109-036-097 or GE Healthcare, Freiburg, Germany, #NA9310V)(30 min., RT) and 50 μl of TMB (10 min., RT) substrate. Color development was stopped by addition of 50 μl of 1 N hydrochloric acid (HCl) and absorbance measured at 450 nm in a plate reader.

1. A method of delivering a pharmaceutically active compound across the blood-brain-barrier in an individual comprising administering to the individual an effective amount of a fusion polypeptide comprising
at least one binding pair, which comprises an antibody heavy chain variable domain and an antibody light chain variable domain, and which binds to an internalizing cell surface receptor, and
at least one pharmaceutically active compound,
whereby the ratio of the EC₅₀-value of the binding pair that binds to an internalizing cell surface receptor determined at pH 5.5 and the EC₅₀-value of the same binding pair to the same receptor determined at pH 7.4 is 10 or more, such that the fusion polypeptide delivers the pharmaceutically active compound across the blood-brain-barrier.

2. (canceled)

3. A method of transcytosing epithelial cells of a subject comprising administering to the subject a fusion polypeptide comprising
at least one binding pair, which comprises an antibody heavy chain variable domain and an antibody light chain variable domain, and which binds to an internalizing cell surface receptor, and
at least one pharmaceutically active compound,
whereby the ratio of the EC₅₀-value of the binding pair that binds to an internalizing cell surface receptor determined at pH 5.5 and the EC₅₀-value of the same binding pair to the same receptor determined at pH 7.4 is 10 or more.

4. A fusion polypeptide comprising
at least one binding pair, which comprises an antibody heavy chain variable domain and an antibody light chain variable domain, and which binds to an internalizing cell surface receptor, and
at least one pharmaceutically active compound,
whereby the ratio of the EC₅₀-value of the binding pair that binds to an internalizing cell surface receptor determined at pH 5.5 and the EC₅₀-value of the same binding pair to the same receptor determined at pH 7.4 is 10 or more.

5. A method of increasing transport of at least one pharmaceutically active compound across the blood-brain-barrier in an individual relative to the transport across the blood-brain-barrier of an unconjugated form of the one or more pharmaceutically active compound, comprising administering to the individual an effective amount of a fusion polypeptide comprising
at least one binding pair, which comprises an antibody heavy chain variable domain and an antibody light chain variable domain, and which binds to an internalizing cell surface receptor, and
the at least one pharmaceutically active compound,
whereby the ratio of the EC₅₀-value of the binding pair that binds to an internalizing cell surface receptor determined at pH 5.5 and the EC₅₀-value of the same binding pair to the same receptor determined at pH 7.4 is 10 or more, such that the fusion polypeptide transports the pharmaceutically active compound across the blood-brain-barrier.

6. A method of selecting a binding pair for use in efficient blood-brain-barrier transport of one or more pharmaceutically active compounds comprising measuring a ratio of the EC₅₀-values for binding of one or more binding pairs to an internalizing cell surface receptor at pH 5.5 and pH 7.4, and selecting one or more binding pairs wherein the ratio is 10 or more.

7. (canceled)

8. The method of claim 20, characterized in that the CNS-related disease is selected from (i) neurodegenerative diseases or disorders selected from Parkinson's disease, Alzheimer's disease, and Huntington's disease; (ii) psychiatric diseases selected from depression, anxiety disorders, and schizophrenia; (iii) neuroinflammatory and other neurological disorders selected from multiple sclerosis, Amyotrophic Lateral Sclerosis, autism, and pain; (iv) tumors of the CNS, and (v) viral and bacterial infections of the CNS.

9. The method, fusion polypeptide or pharmaceutical formulation of any one of claims 1, 3-6 and 19 wherein the ratio is selected from about 15 and 15 or more, or wherein the EC₅₀-value determined at pH 5.5 is 1000 ng/ml or more.

10. (canceled)

11. (canceled)

12. The method, fusion polypeptide or pharmaceutical formulation of any one of claims 1, 3-6 and 19, wherein the binding pair is selected from an Fv, a Fab, a Fab′, a Fab′-SH, a F(ab′)₂, diabody, a linear antibody, a single-chain antibody, a multispecific antibody, a full length heavy chain, a full length light chain, a complete antibody, a bispecific antibody, a trispecific antibody, a tetraspecific antibody, and a hexaspecific antibody.

13. The method, fusion polypeptide or pharmaceutical formulation of any one of claims 1, 3-6 and 19, wherein the pharmaceutically active compound is attached to the at least one binding pair by a linker, or wherein the pharmaceutically active compound is directly fused to the at least one binding pair.

14. (canceled)

15. The method, fusion polypeptide or pharmaceutical formulation of any one of claims 1, 3-6 and 19, wherein the internalizing cell surface receptor is selected from a sialoglycoprotein receptor, an alpha(2,3)sialoglycoprotein receptor, a diphtheria toxin receptor a heparin-binding epidermal growth factor-like growth factor, a folate receptor, a glutamate receptor, a glutathione receptor, an insulin receptor, an insulin-like growth factor receptor, a leptin receptor, a low-density lipoprotein receptor, an LDL-related protein 1 receptor, an LRP2 receptor, an LRP4 receptor, an LRP5 receptor, an LRP6 receptor, an LRP8 receptor, a mannose 6-phosphate receptor, a scavenger receptor (class A or B, types I, II or III, or CD36 or CD163), a substance P receptor, a thiamine transporter, a transferrin-1 receptor, a transferrin-2 receptor, and a vitamin B12 receptor, or wherein the internalizing cell surface receptor is a transferrin receptor.

16. (canceled)

17. The method, fusion polypeptide or pharmaceutical formulation of any one of claims 1, 3-6 and 19, wherein the pharmaceutically active compound is selected from a drug, a label, a cytotoxin, an enzyme, a growth factor, a transcription factor, a radionuclide, a ligand, a liposome, a nanoparticle, a viral particle, a cytokine, and an antibody or active fragment thereof.

18. (canceled)

19. A pharmaceutical formulation comprising the fusion protein of claim 4, optionally with a pharmaceutically acceptable carrier or with at least one additional therapeutic agent.

20. A method of treating a CNS-related disease using the pharmaceutical formulation of claim 19.
